(19) United States
(12) Patent Application Publication
(10) Pub. No.: US 2025/0354127 A1
(43) Pub. Date: Nov. 20, 2025

(54) CRYOPRESERVED DAIRY GOAT SEMEN METABOLITE SCREENING AND DILUENT PREPARATION METHOD

(71) Applicants: NORTHWEST A&F UNIVERSITY, XIANYANG (CN); SHAANXI SHENGQUAN DAIRY TECHNOLOGY CO., LTD., XIANYANG (CN)

(72) Inventors: HUAIPING SHI, XIANYANG (CN); YIHUA SHI, XIANYANG (CN); JINTAO LI, XIANYANG (CN); KANGQI YANG, XIANYANG (CN); JUN LUO, XIANYANG (CN); CONG LI, XIANYANG (CN); JIE LI, XIANYANG (CN); JIANHONG HU, XIANYANG (CN); WEI WANG, XIANYANG (CN)

(73) Assignees: NORTHWEST A&F UNIVERSITY, XIANYANG (CN); SHAANXI SHENGQUAN DAIRY TECHNOLOGY CO., LTD., XIANYANG (CN)

(21) Appl. No.: 19/015,631

(22) Filed: Jan. 10, 2025

(30) Foreign Application Priority Data
May 16, 2024 (CN) 202410610129.8

(51) Int. Cl. C12N 5/00; C12N 5/071

(52) U.S. Cl. CPC C12N 5/52; C12N 5/0612

(57) ABSTRACT

A cryopreserved dairy goat semen metabolite screening and diluent preparation method: preserve semen with base diluent until the day 5, select samples on the days 0, 1, 3 and 5 for metabolome sequencing, screen out gamma-aminobutyric acids decreasing significantly with the preservation time, add different concentrations of gamma-aminobutyric acids to detect sperm viability and motility, and determine the optimal concentration of gamma-aminobutyric acids. The optimal addition concentration of gamma-aminobutyric acids is 1.0 g/L, ensuring that the viability and motility of sperms preserved until the day 5 reach 56.49% and 53.69% and that plasma membrane integrity and acrosome integrity reach 58.93% and 60.12%, and effectively improving the oxidation resistance of sperms and reduce oxidative damage. The invention effectively improves the semen cryopreservation effect, has important significance for reducing breeding stock and improving artificial insemination and reproductive efficiencies in the animal husbandry, high popularization and application prospects and economic values.

### TECHNICAL FIELD

The invention belongs to the technical field of animal reproduction, in particular to cryopreserved dairy goat semen preservation and diluent preparation and application.

### BACKGROUND ART

Dairy goats are important milk yielding animals in the development of characteristic dairy products in China, and goat milk has attracted much attention because of its high nutritional value. At present, the traditional dairy goat breeding mode cannot meet the industrial development requirements. To actively promote the scientific, large-scale and modern development of dairy goat breeding, it is necessary to improve and optimize the existing animal breeding technology. Semen preservation technology is the key link of artificial insemination technology, and dairy goat sperms are more likely to die in vitro compared with other animal sperms. Therefore, the research on cryopreserved dairy goat semen diluent is of great significance for promoting artificial insemination technology, improving the utilization rate of excellent tupping rams and prolonging the service life of the tupping rams.

Basic theoretical basis of semen cryopreservation: semen is preserved at 0-5 degrees C. after being diluted with a special diluent; in this environment, the movement of sperms can be properly slowed down, and the metabolism can be inhibited, thus reducing the energy consumption of sperms and ensuring that the sperms are viable for a longer time and have fertility. Semen cryopreservation is achieved by the following ways: first, reduce the sperm survival environment temperature, ensure that the sperms are in a semi-dormant state, and reduce energy consumption; second, properly supplement some energy substances such as glucose and fructose to maintain the consistency between the internal and external environments for the sperms; third, exogenously add antioxidant substances or antibiotics, reduce the oxidative damage to the sperms and ensure that the semen is in an antibacterial environment; fourth, regulate the pH of the semen, ensure that the sperms are in a weak acid state by adding Tris, sodium citrate and other substances, keep consistent with the physiological environment in the male animal before, and reduce the stress response caused by environmental changes.

Gamma-aminobutyric acid, a compound with the chemical formula C₄H₉NO₂, alias 4-aminobutyric acid (γ-aminobutyric acid, GABA for short), is an amino acid found widely in vertebrates, plants and microorganisms. Gamma-aminobutyric acid is an important inhibitory neurotransmitter in the central nervous system, with good water solubility and thermal stability. It is found that GABA has edible safety as a non-protein amino acid with low molecular mass and can be used in the production of foods such as beverages. Studies have shown that a certain amount of GABA has physiological effects on improving sleep quality and lowering blood pressure.

Previous researches and patents mainly focused on environmental additive buffer substances (such as Tris and sodium citrate) and nutritional substances (sugars) required for sperm survival, and had no detailed studies on the changes of small molecule substances in semen preservation. Therefore, on the basis of ensuring the basic conditions of in vitro semen preservation, new research needs to explore the changes of small molecular substances in semen in different time periods, search for small molecular substances required for in vitro sperm preservation, and add them into semen diluent to gradually approach the environment required for in vivo sperm survival and prolong the in vitro sperm preservation time, which is of great significance for improving the reproductive efficiency of dairy goats and promoting the development of dairy goat industry.

### SUMMARY OF THE INVENTION

The invention aims to provide a cryopreserved dairy goat semen metabolite screening and diluent preparation method.

To achieve the above purpose, the invention adopts the following technical proposal:
1) prepare base diluent: add 30.3 g/L Tris, 16.0 g/L citric acid, 6.4 g/L glucose, 6.4 g/L D-fructose, 2.5 g/L trehalose, 1.0 g/L BSA and 3.75 g/L soybean lecithin into distilled water to 1 L, dissolve in a water bath for 1 hour at 37 degrees C., filter and sterilize, and then add 1 million IU/L penicillin and 1 million IU/L streptomycin;
2) mix dairy goat semen with the diluent, and conduct gradient cooling to 4 degrees C. for preservation;
3) collect samples in 2 mL cryogenic tubes on the days 0, 1, 3 and 5 of semen preservation for metabolome sequencing;
4) add gamma-aminobutyric acids into the base diluent for semen cryopreservation, and detect the semen quality in different storage periods of time.

In the step 2), the mixing temperature of semen and diluent should be maintained at 35-37 degrees C., the dilution factor is 8, and the gradient cooling rate is 8-10 degrees C./h.

In the step 4), the concentration of gamma-aminobutyric acids is 1.0 g/L, and the changes of sperm viability, motility, plasma membrane integrity, acrosome integrity and various antioxidant indexes are evaluated.

The invention has the following beneficial effects:
the invention mainly improves the semen preservation diluent by screening the changes of small molecular substances during dairy goat semen cryopreservation; metabolome sequencing is performed on semen with different preservation time periods, and key substances decreasing with the preservation time is screened and added into the semen preservation diluent, breaking the traditional diluent method that the nutrient content is changed only by adjusting different antioxidant substances; small molecular substances that are rapidly consumed in semen preservation can be effectively screened out, and then the semen preservation diluent is gradually improved in order to achieve an environment closer to the in vivo semen survival and increase the in vitro sperm preservation time; therefore, the invention can not only improve the semen preservation time of the dairy goats, but also be applied to other livestock, has important significance for reducing breeding stock, improving artificial insemination and reproductive efficiencies in animal husbandry, and has high popularization and application prospects and economic values.

### BRIEF DESCRIPTION OF THE DRAWINGS

FIG. 1 shows differential metabolites in the top 20 VIP values of A/D groups in a positive ion mode.

FIG. 2. shows the effects of gamma-aminobutyric acid addition on sperm plasma membrane integrity (a) and acrosome integrity (b).

FIG. 3. shows the effects of gamma-aminobutyric acid addition on antioxidant indexes of sperms.

### DETAILED DESCRIPTION OF THE INVENTION

The technical proposal in the embodiments of the invention is clearly and completely described below in combination with the drawings in the embodiments of the invention. Obviously, the described embodiments are only part of the embodiments of the invention, but not all the embodiments. Based on the embodiments of the invention, all other embodiments obtained by ordinary technicians in the field without creative labor fall within the scope of protection of the invention.

### Embodiments

### (I) Critical Metabolite Screening of Cryopreserved Dairy Goat Semen

1) prepare base diluent: add 30.3 g/L Tris, 16.0 g/L citric acid, 6.4 g/L glucose, 6.4 g/L D-fructose, 2.5 g/L trehalose, 1.0 g/L BSA and 3.75 g/L soybean lecithin into distilled water to 1 L, dissolve in a water bath for 1 hour at 37 degrees C., filter and sterilize, and then add 1 million IU/L penicillin and 1 million IU/L streptomycin; select semen with motility more than 80% for preservation, and keep diluent warm in a water bath at 37 degrees C. before semen dilution; gradually dilute at the dilution ratio (add the diluent into each group of semen along the tube wall during the operation), dilute the semen with the diluent at 1:1 and stand for 3 minutes, then dilute the semen with the diluent at 1:3 and stand for 3 minutes, and let the ratio of semen to diluent reach 1:7 (keep the temperature at 33-35 degrees C. during dilution); bind a centrifuge tube containing the diluted semen with a rubber band and put the centrifuge tube into a beaker containing isothermal water, keep the centrifuge tube vertical and the water level in the beaker higher than the diluent level of in the centrifuge tube, place for 1 hour at room temperature, put the beaker into a refrigerator at 4 degrees C., keep the cooling rate at 8-10 degrees C./h, and shake the centrifuge tube every 8 hours to prevent the sperms from accumulating, sinking to bottom and resulting in death;

2) take 5 μL of semen on the days 0, 1, 3 and 5 of semen preservation, put the semen on slides, preheat an objective table and perform microscopic examination at 37 degrees C., and detect semen motility and viability by Meilan CASA semen assisted analysis system; select at least five random fields of view for determination, with a minimum semen count of 200 in each field; the ratio of number of live sperms to total number of sperms is sperm viability, and the ratio of number of linearly moving sperms to total number of sperms is sperm motility. Average the results, and record the test data (see Table 1);

TABLE 1
Changes of sperm viability and motility
in different preservation periods of time

| | Preservation days/d | | | |
|---|---|---|---|---|
| Item | 0 | 1 | 3 | 5 |
| Sperm viability | 85.33 ± 1.24 | 81.65 ± 1.63 | 70.88 ± 2.11 | 53.42 ± 1.28 |
| % | | | | |
| Sperm motility | 82.64 ± 1.05 | 76.67 ± 2.25 | 67.36 ± 1.77 | 50.02 ± 1.36 |
| % | | | | |

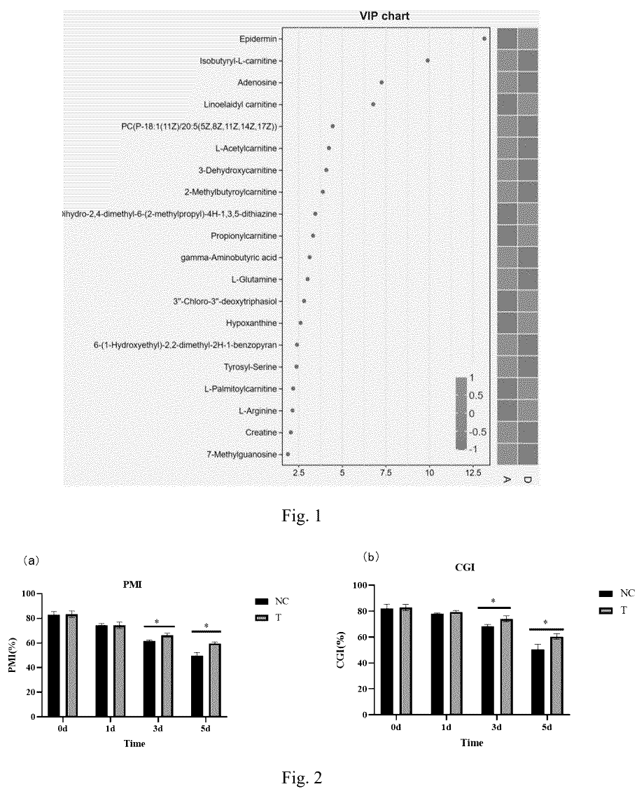

3) collect samples in 2 mL cryogenic tubes on the days 0, 1, 3 and 5 of semen preservation and extract metabolites by the following method: transfer 100 μL of samples to a centrifuge tube and add 400 μL of extracting solution containing isotopically labeled mixture (the ratio of methanol to acetonitrile ratio is 1:1); use a centrifuge to mix sample mixture for 30 seconds; apply ultrasound in an ice-water bath for 10 minutes; let the sample stand for 1 hour at −40 degrees C.; then put the sample into the centrifuge at 4 degrees C., and centrifuge at 12,000 rpm for 15 minutes; finally, take and add supernatant into 2 mL sample vial for testing; metabolome sequencing was performed by Guangzhou GENE DENOVO Biotechnology Co., Ltd. to screen out critical metabolites. The sequencing results are shown in FIG. 1.

(II) Research on Effects of Adding Gamma-Aminobutyric Acids into Semen Diluent on Cryopreservation of Dairy Goat Semen

1) add different concentrations of gamma-aminobutyric acids into a tupping ram semen base diluent, and take a group without gamma-aminobutyric acid added as the control group; take a group with gamma-aminobutyric acid added as the experimental group, the addition concentrations are respectively 0.5 g/L, 1.0 g/L, 1.5 g/L and 2.0 g/L, and six duplications exist in each group; screen out the optimal concentration of gamma-aminobutyric acid according to semen quality indexes, set the optimal concentration to T group, and detect the semen membrane and acrosome integrity, various antioxidant enzyme activities and ROS content change after adding the optimal concentration of gamma-aminobutyric acid;

Table 2 shows that sperm viability and motility were higher than those in other addition groups when the concentration of gamma-aminobutyric acid was 1 g/L, indicating that gamma-aminobutyric acids can improve the semen preservation effect;

TABLE 2
Effects of different concentrations of gamma-aminobutyric
acids on sperm viability and motility in rams

| γ-amino- | | | | |
|---|---|---|---|---|
| butyric | Sperm viability % | | Semen motility % | |
| acid (g/L) | Day 0 | Day 5 | Day 0 | Day 5 |
| 0 | 84.12 ± 2.15 | 51.33 ± 1.56^{b} | 81.36 ± 2.05 | 50.15 ± 1.25^{b} |
| 0.5 | 84.12 ± 2.15 | 52.45 ± 1.22^{b} | 81.36 ± 2.05 | 50.69 ± 1.05^{b} |
| 1.0 | 84.12 ± 2.15 | 56.49 ± 1.32^{a} | 81.36 ± 2.05 | 53.69 ± 0.84^{a} |
| 1.5 | 84.12 ± 2.15 | 50.52 ± 2.74^{b} | 81.36 ± 2.05 | 47.32 ± 2.41^{c} |
| 2.0 | 84.12 ± 2.15 | 45.36 ± 2.31^{c} | 81.36 ± 2.05 | 45.55 ± 1.52^{d} |

Note:
Different lowercase letters indicate significant differences in values in the same column.

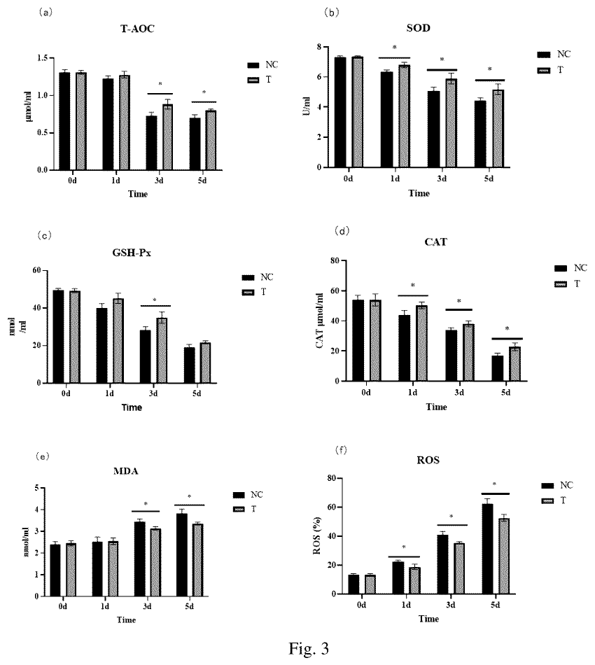

2) detect the plasma membrane integrity (PMI) of tupping ram sperms preserved at 4 degrees C. for 0, 1, 3 and 5 days by SYBR-14/PI fluorescence staining; detect the sperm acrosome integrity (CGI) by FITC-PNA fluorescence staining, with kits from Shanghai GENMED Pharmaceutical Technology Co., Ltd. The results showed (see FIG. 2) that the plasma membrane integrity and acrosome integrity of sperms in T group were significantly higher than those in NC group on the day 3 of preservation (P<0.05), and the plasma membrane integrity and acrosome integrity of sperms in the experimental group reached 58.93% and 60.12% respectively on the day 5 of preservation;

3) antioxidant indexes determined by Solarbio kits include T-AOC, SOD, GSH-Px, MDA and CAT; the results showed (see FIG. 3) that on the day 1 of preservation, the activities of SOD and CAT enzymes in T group were significantly increased (P<0.05) than that of NC group, MDA, GSH-Px and T-AOC were not significantly different, and the level of ROS was significantly decreased (P<0.05); the levels of T-AOC, SOD, GSH-Px and CAT in T group were significantly higher than that in NC group (P<0.05), and the levels of MDA and ROS in T group were significantly lower than that in NC group (P<0.05); these results indicate that gamma-aminobutyric acids can effectively enhance the oxidation resistance of sperms and reduce the oxidative damage.

1. A cryopreserved dairy goat semen metabolite screening and diluent preparation method, characterized by including the following steps:
1) prepare base diluent: add 30.3 g/L Tris, 16.0 g/L citric acid, 6.4 g/L glucose, 6.4 g/L D-fructose, 2.5 g/L trehalose, 1.0 g/L BSA and 3.75 g/L soybean lecithin into distilled water to 1 L, dissolve in a water bath for 1 hour at 37 degrees C., filter and sterilize, and then add 1 million IU/L penicillin and 1 million IU/L streptomycin;
2) mix dairy goat semen with the diluent, and conduct gradient cooling to 4 degrees C. for preservation;
3) collect samples in 2 mL cryogenic tubes on the days 0, 1, 3 and 5 of semen preservation for metabolome sequencing;
4) add gamma-aminobutyric acids into the base diluent for semen cryopreservation, and detect the semen quality in different storage periods of time.

2. The cryopreserved dairy goat semen metabolite screening and diluent preparation method according to claim 1, characterized in that in the step 2), the mixing temperature of semen and diluent should be maintained at 35-37 degrees C., the dilution factor is 8, and the gradient cooling rate is 8-10 degrees C./h.

3. The cryopreserved dairy goat semen metabolite screening and diluent preparation method according to claim 1, characterized in that in the step 4), the concentration of gamma-aminobutyric acids is 1.0 g/L, and the changes of sperm viability, motility, plasma membrane integrity, acrosome integrity and various antioxidant indexes are evaluated.
